(19) United States
(12) Patent Application Publication
(10) Pub. No.: US 2006/0240555 A1
(43) Pub. Date: Oct. 26, 2006

(54) Fibrin cell supports and method of use thereof

(75) Inventor: Vincent Ronfard, Newton, MA (US)

Correspondence Address: FULBRIGHT & JAWORSKI L.L.P., 600 CONGRESS AVE., SUITE 2400, AUSTIN, TX, 78701, US

(73) Assignee: DFB Pharmaceuticals, Inc., Fort Worth, TX (US)

(21) Appl. No.: 11/362,379

(22) Filed: Feb. 24, 2006

Related U.S. Application Data
(63) Continuation of application No. 10/655,435, filed on Sep. 4, 2003, now abandoned.
(60) Provisional application No. 60/408,566, filed on Sep. 6, 2002.
(60) Provisional application No. 60/433,715, filed on Dec. 16, 2002.

(51) Int. Cl. C12N 5/02; C12N 5/00

(52) U.S. Cl. 435/395; 435/325

(57) ABSTRACT

The present invention relates to fibrin cell supports for cell cultures formed by the mixture of plasma proteins including fibrinogen and thrombin. The fibrin cell supports are preferably used for preparing a culture of cells such as keratinocytes, recovering the culture in the form of a reconstituted tissue, and transporting same. The reconstituted tissue is particularly suitable for use as a skin graft.

### RELATED APPLICATIONS

This application claims priority to U.S. Application Nos. 60/408,566, filed Sep. 6, 2002; and 60/433,715, filed Dec. 16, 2002, each of which are incorporated herein by reference in their entireties.

### FIELD OF THE INVENTION

The present invention relates to a fibrin cell support for cell cultures, containing a coagulated mixture of plasma proteins including fibrinogen and thrombin; its use in the preparation of cell cultures; its transport and transplantation in the form of an isolated cell, colonies of cells, or a reconstituted epithelia; and it's use for therapeutic purposes.

### BACKGROUND OF THE INVENTION

The reconstitution of a living skin similar to the human skin from a few cells obtained from a biopsy, or of a simplified skin performing the physiological functions of a normal skin, is being studied extensively with the aim of replacing skin damaged by disease (hemangiomas, keloids, hypertrophic scarring, bullous pemphigoid, viral or bacteial infection, or acne), by trauma such as major burns, by an acute wound (such as plastic surgery or removal of a tumor mass) or by chronic wounds (e.g., ulcers).

The skin is a complex organ composed of three juxtaposed tissues: the epidermis, 85% of which is constituted by keratinocytes, which form the impermeable layer that isolates the body from the outside environment; the dermis, which comprises cells, including fibroblasts, separated by a connective tissue composed mainly of collagen; and the hypoderma, which includes the cells dedicated to storing fats. Artificial reconstitution of such a complex organ thus poses numerous problems. Both dermis and epidermis have been partially reconstituted in vitro. (See Bell et al., Proc. Natl. Acad. Sci. 76:1979-1274).

Starting with skin biopsies, fibroblasts have been successfully established in cultures, first in monolayers, then, after a number of passages, by dispersing these cells in culture medium containing collagen (extracted from rat's tail tendons), the latter forming a gel and permitting three-dimensional cultures. In such cultures, the fibroblasts interact with the collagen matrix, organizing and contracting it, as occurs in a normal dermis. This tissue, reconstituted in vitro, is known as a “dermal equivalent”. After a few weeks' growth, the mechanical qualities of the equivalent dermis allow it to be used for grafting onto a patient or injured person. It does not appear to be rejected by its host. However, this equivalent dermis is merely a temporary dressing as it cannot restore the skin's cutaneous barrier function.

Furthermore, a method and a culture medium enabling keratinocytes to be grown for long periods has been developed previously. (See Green et al. (1979) Proc. Natl. Acad. Sci. 76:5665-8). This method includes the step of inoculating the keratinocytes dispersed with trypsin on a preestablished monolayer of fibroblasts, in particular 3T3 cells, which have been lethally irradiated and which serve as a nutritive layer and as a matrix. An epidermal layer develops very rapidly to form a tissue having a thickness of 3 to 5 cells, and it can be grafted onto a patient and continue to differentiate in situ. This technique has been used to treat patients suffering from severe burns. (See Gallico et al. (1984) New England J. Med. 311:448).

Using the technique of Green et al., it is possible to obtain, from a biopsy of two square centimeters, an epidermis of one square meter in the space of three weeks.

However, recovery of the reconstituted tissue in order to make a graft therefrom still poses a number of technical problems. For example, it is necessary to detach the multilayered epithelium from the culture dish using an enzyme treatment without dissociating the cells. During this procedure, a retraction of the cell layer, and, hence, a loss of a certain percentage of the surface area of the graft, is observed. Moreover, once the reconstituted tissue has been detached, it has to be fastened to a support that enables it to be transported and grafted onto the patient. Typically, an adhesive-treated gauze dressing is generally used. These manipulations are both delicate and time consuming, which jeopardizes the quality of the graftable epithelium.

Thus, it would thus be highly beneficial to have at one's disposal novel fibrin cell supports that can be resorbed by the patient who has received the graft and that simplify the handling and preserving the quality of the cultured epithelium. In addition, to ensure their availability, such supports or their constituents should lend themselves to preparation and packaging in accordance with industrial processes.

### SUMMARY OF THE INVENTION

In one aspect, the invention involves a fibrin cell support for cell cultures including thrombin and fibrinogen, where the concentration of thrombin is between about 0.5 U/ml and about 2.5 U/ml. Generally, the concentration of fibrinogen is between about 10 and about 250 mg/ml. In some embodiments of the present invention, the fibrin cell support includes a protease inhibitor (i.e., an anti-protease) such as aprotinin or a synthetic protease inhibitor (e.g., tranexamic acid), or other molecules including polypeptide growth factors, cytokines, enzymes, hormones, antibiotics, antimycotics, or a combination of two or more of these molecules. In other embodiments of the present invention, the fibrin cell support further includes one or more cells, e.g., keratinocytes or other epithelial cells. In some embodiments, the thrombin is calcic thrombin.

In another aspect, the present invention provides a method of preparing a fibrin cell support, including the steps of mixing equivalent volumes of a first solution comprising fibrinogen and a second solution comprising thrombin; and distributing the mixture onto a surface, such that a fibrin cell support is formed on the surface. In certain embodiments of the present invention, this method further provides contacting with the fibrin cell support one or more cells, e.g., keratinocytes or other epithelial cells.

In yet another aspect, the present invention provides a method of using a fibrin cell support, including the steps of contacting one or more cells with a fibrin cell support to form a skin replacement tissue, where the support includes thrombin and fibrinogen; and recovering, transporting and applying the skin replacement tissue as a graft. The cells may be located on the surface of the fibrin cell support or integrated within the fibrin cell support.

In a further aspect, the present invention provides a method of using a skin replacement tissue, by contacting one or more cells with a solution comprising fibrinogen and thrombin, to form a skin replacement tissue and transporting the skin replacement tissue to a patient in need thereof.

In another aspect, the present invention provides a method for decreasing the probability of mechanical damage to the fibrin cell support during transport prior to transplantation.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE FIGURES

FIG. 1 is a series of photographs that demonstrate the increased in vitro attachment of epithelia on a fibrin support located on the surface of an artificial membrane.

FIG. 2 is a series of photographs that demonstrate the increased in vivo acceptance of a graft that includes a fibrin cell support and cultured epithelia.

FIG. 3 is a graph that demonstrates the reduced wound contraction of an epithelial cell graft when cultured with a fibrin cell support.

### DETAILED DESCRIPTION OF THE INVENTION

The present invention provides novel fibrin cell supports useful for culturing cells. These supports are formed by the coagulation of plasma proteins including fibrinogen in the presence of thrombin. This coagulation is chiefly the result of the formation of a polymerized fibrin network, which imitates the formation of a blood clot. Thrombin converts fibrinogen to fibrin by enzymatic cleavage, and also converts protransglutaminase (factor XIII) to an active transglutaminase (factor XIIIa). Calcium accelerates the proteolytic activity of thrombin. To form a support suitable for the preparation of cell cultures, coagulation is carried out under conditions that are conducive to the formation of a film, and more particularly, in cell culture flasks or dishes.

One aspect of the present invention involves a fibrin cell support useful for culturing cells. This support is generated by combining a solution of a plasma protein such as fibrinogen with a solution of calcic thrombin such that a fibrin matrix forms, on which cells can be supported and cultured. The term “calcic thrombin” as used herein includes thrombin in the presence of calcium. For example, calcic thrombin includes a solution containing thrombin and any concentration of calcium. Fibrinogen can also be contacted with thrombin in the absence of calcium.

Plasma proteins such as fibrinogen can be obtained from human plasma, (e.g., obtained from blood donors) or can be recombinant. If in solid form (such as freeze-dried or lyophilized), the fibrinogen must be reconstituted, e.g., in an isotonic solution. In some embodiments of the present invention, the isotonic solution is isotonic sodium chloride containing calcium chloride. The concentration of sodium chloride may be in the range of about 0.5% to about 5.0%, preferably in the range of about 1.0% to about 3.0%, and the concentration of calcium chloride may be in the range of about 0.5 mM to about 5 mM, preferably in the range of about 1 mM to about 2 mM. The isotonic solution may further comprise one or more protease inhibitors, e.g., a polyvalent protease inhibitor such as aprotinin, provided in a concentration range of about 1,000-10,000 KIU/ml (kallikrein inhibitor units/ml), preferably about 3000 KIU/ml. Alternatively, such protease inhibitor(s) in solution may be added directly to the fibrinogen to reconstitute the protein. The concentration of fibrinogen is usually about 1-1000 mg/ml, preferably 10-250 mg/ml, more preferably 50-150 mg/ml, and most preferably 60 mg/ml. The fibrinogen solution may additionally contain other plasma proteins or polypeptides including, but not limited to, fibronectin, Factor VIII and Factor XIII.

Likewise, the thrombin may also be derived from natural sources or may be recombinant or synthetic. If in solid form, thrombin can be reconstituted in an isotonic solution containing calcium, e.g., 1.1% NaCl containing 1 mM calcium chloride. The concentration of the thrombin solution is usually about 0.1-10 U/ml, preferably 0.5-5.0 U/ml, even more preferably 1-3 U/ml and most preferably 2.5 U/ml. Units of thrombin refer to the activity standard as defined by the NIH standard. One NIH unit corresponds to 1.15 International Units. (See, e.g., Gaffney et al. (1995) J. Thromb. Haemost. 74:900-3). Thrombin may also be combined with fibrinogen in the absence of calcium. However, those skilled in the art will recognize that the presence of calcium accelerates the proteolytic activity of thrombin.

The fibrinogen solution and the thrombin solution are combined (usually in equal volumes) and are distributed to a vessel, such as a tissue culture dish, before clotting occurs. Once clotting occurs, a fibrin cell support or matrix is formed. Alternatively, the two solutions may be injected into a vessel simultaneously using two syringes interconnected by a mixing coupling. Generally, the fibrin matrix formed by the combination of the calcic thrombin and the fibrinogen solutions will be transparent. The volume of the solution containing fibrinogen and thrombin used is dependent upon the thickness of the fibrin cell support desired. Typically, about 2.5 ml of each solution is used for approximately every 100 cm² of surface.

Those skilled in the art will recognize that other polypeptides or molecules (e.g., growth factors or cytokines such as EGF, VEGF, PDGF, NGF, and TGF-β; healing agents; enzymes such as matrix-degrading enzymes and matrix-degrading enzyme inhibitors (e.g., TIMPs); antibiotics; and/or and antimycotics) may be added to the fibrinogen solution and/or the thrombin solution prior to, concomitant with, or following the mixing of the calcic thrombin and the fibrinogen components. Moreover, the plasma transglutaminase factor XIIIa may be added to the fibrinogen solution, the thrombin solution, the mixture, or to the fibrin cell support in order to covalently crosslink the resulting fibrin cell support.

The fibrin cell support according to the invention is advantageous when preparing cell cultures, particularly keratinocyte cultures, such as human keratinocyte cultures. These cell cultures can be either primary cultures derived from skin biopsies obtained from a patient that have undergone between 1 and 6 or more passages in 1/15 to 1/20 dilutions, or cells preserved in the form of banks in liquid nitrogen. Cells may be cultured in the presence of a feeder cell layer, such as a layer of lethally-irradiated human fibroblasts (See Limat et al., 1986 J Invest Dermatol. October 1986;87(4):485-8).

In some embodiments of the present invention, cells such as keratinocytes are grown to confluence, trypsinized, suspended in an appropriate culture medium, and replated on the fibrin cell support. Cells may also be plated on the fibrin cell support at a subconfluent density and allowed to reach confluence in culture on the fibrin cell support. Cells may also be added to the mixture of thrombin and fibrinogen prior to coagulation, such that the cells are embedded within the fibrin cell support.

The invention includes any mammalian or non-mammalian mammalian cell type that is capable of being maintained under cell culture conditions, and preferably of in vitro expansion in number and of subsequent integration into a reconstituted epithelium. In some embodiments, the cells are isolated from and re-introduced into the same animal (autologous cells, i.e., cells obtained from the intended recipient), thus avoiding the risk of immune rejection and disease transmission. In other embodiments, the cells are isolated from allogeneic embryonic or neonatal tissue that is inherently less immunogenic than adult tissue. In still other embodiments, so-called immunologically neutral allogeneic cells are used. Immunologically neutral allogeneic cells are allogeneic cells of either fetal or adult origin which themselves have been genetically modified to eliminate the synthesis and/or expression of the cell surface antigens which are responsible for the self/non-self recognition by the immune system of the recipient. Such antigens fall chiefly within, but are not limited to, the major histocompatibility complex (“MHC”), Classes I and II.

Isolated cells can be obtained from humans or other mammals (e.g., rodents, primates, cows, or pigs). In certain embodiments, these cells can be derived from skin or other organs such as eyes, heart, brain or spinal cord, liver, lung, kidney, pancreas, bladder, bone marrow, spleen, muscle, intestine, or stomach. In other embodiments, these cells can be stem cells which can be differentiated into a desired cell type in culture.

In particular embodiments of the invention, the isolated cells are keratinocytes, e.g., epidermal keratinocytes; oral and gastrointestinal mucosal epithelia; urinary tract epithelia; corneal epithelial cells; corneal epithelial stem cells; as well as epithelia derived from other organ systems, skeletal joint synovium, periosteum, bone, perichondrium, and cartilage; fibroblasts; muscle cells (e.g. skeletal, smooth, or cardiac muscle cells); endothelial cells; pericardial cells; dural cells; cells of the meninges; keratinocyte precursor cells; keratinocyte stem cells (e.g., NIKSTh); endothelial cells; pericytes; glial cells; neural cells; amniotic and placental membrane cells; stem cells; and serosal cells (e.g., serosal cells lining body cavities). The cells of the present invention may also include recombinant or genetically modified cells.

Those skilled in the art will recognize that the use of the fibrin cell support according to the invention can be adapted in multiple ways. For example, according to one method of use, the fibrin cell support is prepared in the form of a film, by mixing its two constituents (thrombin, calcic thrombin and fibrinogen) in a culture dish. A suspension of cells is then seeded on this film, in an appropriate culture medium. When the cell culture has become confluent or semi-confluent, it forms a replacement tissue that can be recovered directly as a graft, which can be detached using forceps and transported from the culture dish to the patient. It can be applied to the wound as is, without any need for a temporary support, such as gauze. This method leads to a considerable saving in working time as well as a 100% recovery of the tissue grown.

According to another method of using the fibrin cell support of the invention, the two constituents of the support are mixed with a cell suspension in such a way as to integrate the cells within the film that is subsequently formed. According to this method, the two constituents can be mixed with the cell suspension in a culture dish and then used as a graft, as described above. This method may also be carried out directly on a wound site on a patient, which has been prepared to receive a graft, by spraying a mixture of the fibrin cell support and the cells onto the wound using a vector gas (nitrogen) at a pressure of 2 to 2.5 bars, or by applying a paste to the wound.

According to a further method of using the fibrin cell support according to the invention, the two constituents of the support are mixed to form a viscous cell paste to adhere to a wound. Preferably, the resulting paste is both biodegradable and biocompatible. The paste may be applied to the wound as needed, for example, once weekly. Application of the cell paste according to this embodiment facilitates the induction of granulation tissue and the stimulation of wound closure.

According to yet another method of using the support according to the invention, the two constituents are mixed on a layer of cells that has been preestablished in a culture dish. This is done in such a way that the cells become coated with the film that is subsequently formed. In this method the cells can be detached and transported in order to be applied to a wound as a graft.

In certain embodiments of the invention, the support further contains one or more disinfectants, preferably methylene blue, and/or one or more drugs selected from antibiotics, fibrinolytic agents, and biological response modifiers such as cytokines and wound repair promoters. Preferably, these compounds-are included in an amount up to 1% by weight in terms of the total dry weight of fibrin plus thrombin. Examples of suitable fibrinolytic agents include t-PA, μ-PA, streptokinase, staphylokinase, plasminogen and the like, which promote fibrinolysis and, thus, can be used to control the rate of the degradation of the fibrin film in vivo. As used herein, the term “biological response modifiers” refers to substances that are involved in modifying a biological response, such as wound repair, in a manner which enhances a desired therapeutic effect of the fibrin cell support. Examples of suitable biological response modifiers include cytokines, growth factors, wound repair promoters, and the like.

Additionally, it may be necessary to deliver the fibrin cell support from the facility where it is generated to a facility where it is used. Therefore a system is needed for the transport of the fibrin cell supports of the invention. A first system involves contacting the fibrin cell support with a cylindrical inner carrier. For example, the fibrin cell support can be rolled around the inner carrier, which can be solid or hollow, such as a plastic tube. The fibrin cell support and the inner carrier are then placed in a hollow outer carrier which has an inner diameter larger than the outer diameter of the inner carrier. The space between the fibrin cell support and the inner wall of the outer carrier is then filled with cell media or other suitable material and the outer carrier is sealed so as to maintain a sterile environment.

A second transport system involves contacting the fibrin cell support with an essentially flat or planar carrier (e.g., a natural or synthetic material such as a polyester membrane). The carrier has two sheets operably linked to each other. The carrier can also be an envelope into which the fibrin cell support and suitable cell culture media are inserted. The fibrin cell support is adhered to the carrier with the cells in contact with the carrier. Alternatively, the fibrin cell support is adhered to the carrier with the cells not in contact with the carrier. Suitable carriers may consist of a synthetic membrane made from one or more of the following materials (polyester, PTE or polyurethane); from one or more biodegradable polymers (e.g., hyaluronic acid, polylactic acid or collagen); or a silicone or vaseline gauze dressing, or any other material suitable for wound dressing.

The invention will be further described in the following examples, which do not limit the scope of the invention described in the claims.

### EXAMPLES

### Example 1

### Preparation of a Fibrin Cell Support for Cell Cultures

A fibrin cell support for cell cultures is prepared by mixing a solution containing fibrinogen and a solution containing calcic thrombin.

Lyophilized fibrinogen (375-575 milligrams) is reconstituted with 5 ml of aprotinin (3,000 KIU/ml; kallikrein inhibitor units/ml) then combined with 5 ml of 2.2% NaCl containing 2 mM calcium chloride. Lyophilized thrombin (225 to 275 milligrams; approximately 2500 International Units) is diluted in 1.1% NaCl containing 1 mM calcium chloride to a final concentration of 0.225 to 0.275 milligrams or 2.5 IU. The solubilized fibrinogen and solubilized thrombin are mixed in a 1:1 ratio and dispensed into a cell culture dish or flask (2.5 mls of the fibrinogen-thrombin mixture per 100 cm² of culture dish surface) to form a fibrin cell support. The fibrin cell support is then covered in cell culture medium.

### Example 2

### Preparing a Keratinocyte Culture on the Fibrin Cell Support

Human keratinocytes originating from a skin biopsy are cultured in the presence of lethally-irradiated or mitomycin C-treated human fibroblasts, or lethally-irradiated or mitomycin C-treated murine feeder cells. (See Limat et al. (1986) J Invest Dermatol. 87(4):485-8; Green et al. (1979) Proc. Natl. Aced. Sci. 76:5665).

A layer of confluent keratinocytes is trypsinized, replaced in suspension in culture medium and seeded at subconfluent density (e.g., in a 1/10 dilution) on a tissue culture dish covered with the fibrin cell support prepared as described in Example 1. The keratinocytes are then allowed to reach confluence, at which point the resulting keratinocyte graft can be used in therapeutic methods. The fibrin cell support of this invention stands up well to handling and does not retract at the time of detachment, which makes it possible to recover 100% of the surface area of the cell layer of the culture.

In another embodiment the keratinocytes of the fibrin cell support are at sub-confluent concentrations. For example, the keratinocytes may be in two or more colonies, each colony containing between about 4 and about 1,000 cells.

The keratinocyte graft can be applied to a patient in need thereof in a method such that the fibrin cell support contacts the patient, or alternatively, the keratinocyte cell layer contacts the patient.

### Example 3

### Recovery of a Pre-Established Cell Layer Using the Fibrin Cell Support

Keratinocytes are inoculated according to Green's conventional method, in a Petri dish covered with a layer of lethally irradiated fibroblasts. (See Green et al. (1979) Proc. Natl. Aced. Sci. 76:5665). When keratinocytes are confluent and formed of several layers of cells, the culture medium is removed, an EDTA solution is added for 1 hour 30 minutes. This is followed by washing twice with PBS. The fibrin cell support prepared as described in Example 1 is then poured directly onto confluent keratinocytes.

Upon coagulation of the fibrin cell support, it can be detached mechanically and used as a graft, as demonstrated in Example 2.

### Example 4

### Incorporation of Keratinocytes into the Fibrin Cell Support

Keratinocytes can be embedded within the fibrin cell support by any of several methods. In a first method, a syringe of solubilized fibrinogen and a syringe of solubilized thrombin containing the keratinocytes in suspension are prepared. These keratinocytes may be taken from a fresh, trypsinized culture or from a bank of cells preserved in liquid nitrogen. The two syringes are interconnected by means of a mixing coupling and the resulting fibrin cell support containing the cells is sprayed onto a tissue culture dish (or onto a wound prepared to receive the graft). In this method, the cells are held within the fibrin cell support during its coagulation. The spraying can be carried out using a vector gas (e.g., nitrogen at a pressure of 2 to 2.5 bars) or any other method known to those skilled in the art. This spraying does not damage the cells or denature the polypeptides, and the cell layer can be observed to reform in culture. These cells should thus multiply normally when the mixture is sprayed, in a very thin layer, directly onto a wound.

In a second method, the solubilized fibrinogen and solubilized thrombin are mixed in a 1:1 ratio, combined with a solution containing keratinocytes, and dispensed into a cell culture dish or flask (2.5 mls of the fibrinogen-thrombin mixture per 100 cm² of culture dish surface) to form a fibrin cell support containing keratinocytes.

### Example 5

### Artificial Dermis and Fibrin Epithelium Combination

The fibrin cell support of the present invention is useful for the early and temporary coverage of acute wounds that are currently treated by the application of an acellular artificial skin (e.g., INTEGRA®, developed by Integra LifeSciences, Inc, Plainsboro, N.J.). Generally, when an acellular artificial skin is applied onto the wound bed of a patient in need thereof, 3 to 5 weeks of time are required for the patient's own cells to invade the matrix and create a new dermis. Thereafter, an epidermal autograft is necessary to achieve permanent wound closure. Conventional cultured epithelium autografts have been partially unsuccessful to provide that coverage, because of lack of epithelial anchorage onto the INTEGRA® matrix. The use of the fibrin cell support of the present invention greatly improves the epithelial anchorage and success of the skin graft.

Keratinocytes were cultured according to the method of Rheinwald & Green modified by Limat and colleagues (Gallico et al. 1984, Limat et al. 1986). Human keratinocytes were cultivated on the top of the fibrin matrix in the presence of aprotinin. When the culture reached confluence, the epithelial sheet along with the fibrin was applied on INTEGRA®. A control culture (CEA control) was treated with Dispase upon reaching confluence in order to detach the epithelial sheet from the bottom of the dish. (See Green et al., 1979). Then the epithelial sheet was lifted off the plate using a silicone membrane, and applied onto the INTEGRA®.

In vitro grafting and culture: INTEGRA® pieces (0.8×0.8 cm) were seeded with human dermal fibroblasts and cultured for 2 weeks. They were then placed on cell culture inserts so that they were fed from the basolateral surface. Control epithelium (CEA) and fibrin epithelium were then placed on the INTEGRA® and covered with a silicone membrane.

In vivo grafting: surgery on mice proceeded according to standard guidelines of animal care and approved by veterinary authorities. Athymic mice (NIH Swiss nude) approximately 6-8 weeks in age (24-32 g in weight) were anaesthetized. A full thickness skin wound approximately 2.2 cm² was made on the central dorsum of each animal and covered with INTEGRA®. Then, Vaseline gauze and adhesive bandages were applied onto the graft. Dressings were changed every week. CEA control and fibrin epithelium were grafted 3 weeks later. One week later, the silicone membrane was removed but the dressing kept. Another week later, all remaining dressing was removed and the grafts were left uncovered. Biopsies were harvested from 15 and 21 days after grafting.

Histology: Biopsies were fixed in buffered formalin (5%) and embedded in paraffin wax five μm sections were stained with haematoxylin-Eosin. Human involucrin (Biomedical Technology Inc., Stoughton, Mass.) was used to identify human keratinocytes from mouse tissue. These studies demonstrate that (1) 100% of the epithelium grown in the presence of the fibrin sheet attached to the INTEGRA® and were able to develop a stratified epithelium after 2 weeks of culture compare to only 50% for the control epithelium; (2) retraction of the grafting area was significantly lower 2 weeks after grafting (p<0.05) when the epithelium was grown in the presence of the fibrin sheet compared to the CEA control epithelium; and (3) 75% of the mice grafted with fibrin epithelium showed the presence of a differentiated human epidermis 21 days after grafting, while such an epidermis was absent in all the animals of the control group (as revealed by the absence of a specific human cell marker). The use of a fibrin matrix also greatly improved cell adhesion, epidermis development and graft take onto INTEGRA®.

Results:

In Vitro Attachment of Epithelia using a Fibrin Cell Support

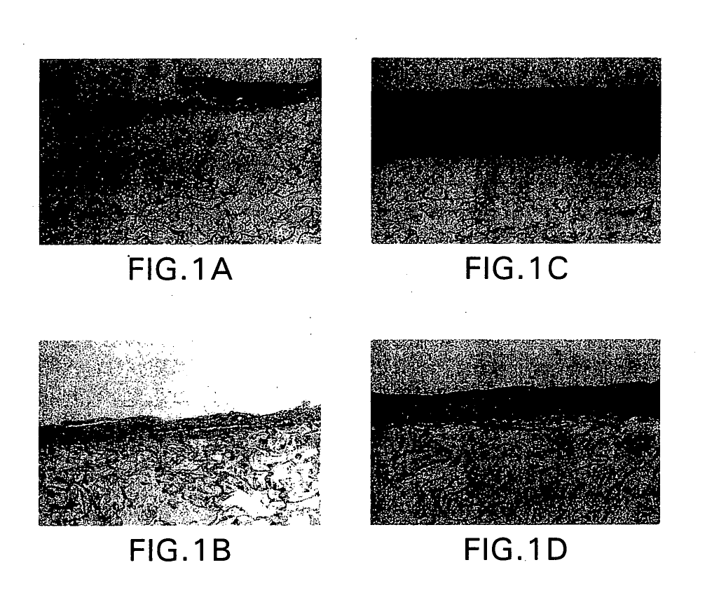

FIG. 1 shows the evaluation of the quality of the epithelium applied on the top of INTEGRA® after 2 days of culture: (A) CEA control on INTEGRA® without fibroblasts, (B) CEA control on INTEGRA® with fibroblasts, (C) fibrin epithelium on INTEGRA® without fibroblasts, (D) fibrin epithelium on INTEGRA® with fibroblasts. A well-organized epithelium is obtained with fibrin epithelium (C, D) when compared to the control CEA (A, B).

Histological cross-sections of the CEA control: showed that: (1) attachment areas were far more numerous when INTEGRA® was populated with cells as compared to unpopulated INTEGRA® matrices and (2) in both the unpopulated INTEGRA® and the INTEGRA® populated with cells, the edges of the epithelium were not attached and were often folded. Until the second day of culture, many areas of the CEA control showed dying cells with apoptotic nucleus and also differentiating cells so unable to further divide. This indicates that the cell take yield for the CEA control on INTEGRA® was poor (FIG. 1).

In contrast, the main attached areas of the fibrin epithelium were at the edges of the grafts (See FIG. 1 C, D). The keratinocytes migrated from the fibrin layer to the INTEGRA®, forming a bond between the fibrin epithelium and the INTEGRA®. The same attachment feature was observed both for fibroblast populated and unpopulated INTEGRA® matrices, creating sufficient attachment strength for the fibrin epithelium to remain in place even when the silicone sheet covering the grafted fibrin epithelium was removed at day 7 of the culture. Close junctions were seen between fibrin layer and fibroblasts in the fibroblasts populated matrices, suggesting the formation of a normal stratified skin architecture. The epithelial layer was made of normal, proliferating cells with round nuclei and no intercellular spaces or intracellular vacuoles, as were seen for the control epithelia. Thus, the fibrin layer provides a suitable environment for the keratinocytes to survive and for the epithelium to stratify. Thus, cell survival after “in vitro grafting” was increased in the presence of the fibrin epithelium in comparison with controls (FIG. 1).

In Vivo Epithelial Graft with a Fibrin Cell Support

A full thickness skin wound approximately 2.2 cm² was made on the central dorsum of each nude mouse and covered with INTEGRA®. CEA control and fibrin epithelium were grafted 3 weeks after the implantation of INTEGRA®. Biopsies were harvested from 15 and 21 days after grafting. Human involucrin antibody was used to differentiate human keratinocytes from mouse tissue by immunohistological staining on histological cross-sections.

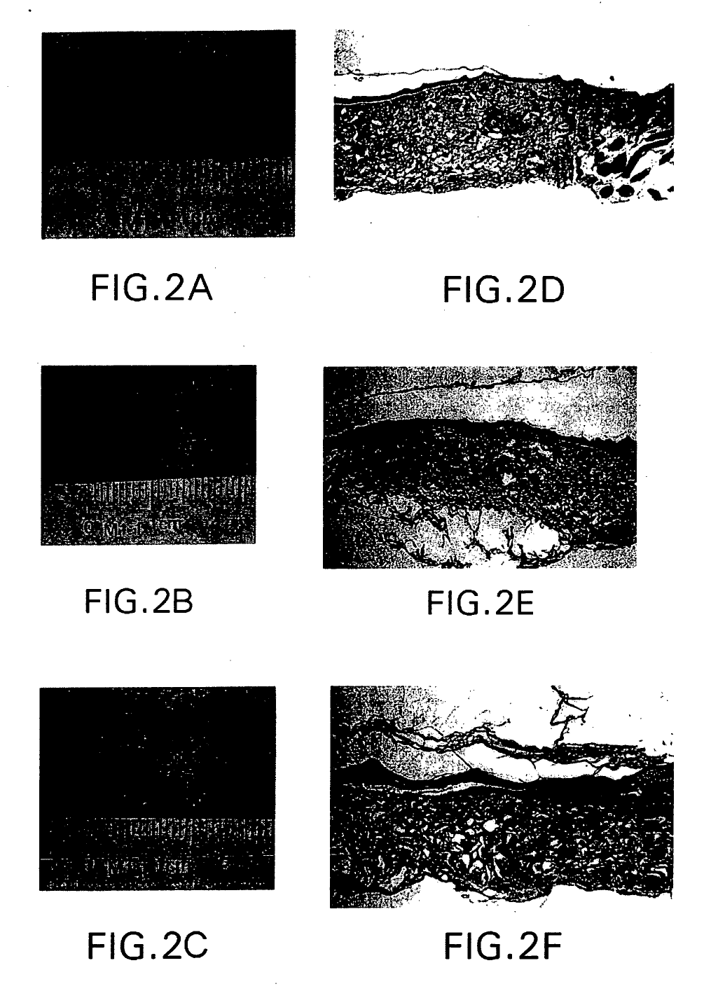

FIG. 2 shows the increased success of an epithelial graft in nude mice using a fibrin cell support. FIG. 2A shows INTEGRA® alone; FIG. 2B INTEGRA® with CEA control; FIG. 2C INTEGRA® with fibrin epithelium and FIGS. 2D-F represent the cross section of each corresponding graft (40× magnification). Substantial graft retraction occurred in the presence of INTEGRA® alone or INTEGRA® with CEA control, as compared to INTEGRA® with a fibrin epithelium. Graft success (or “take”) occurred only when fibrin epithelium was applied onto INTEGRA® as shown by the presence of the human keratinocyte marker, involucrin. The hollow arroweads in FIG. 2F indicate mouse epidermis.

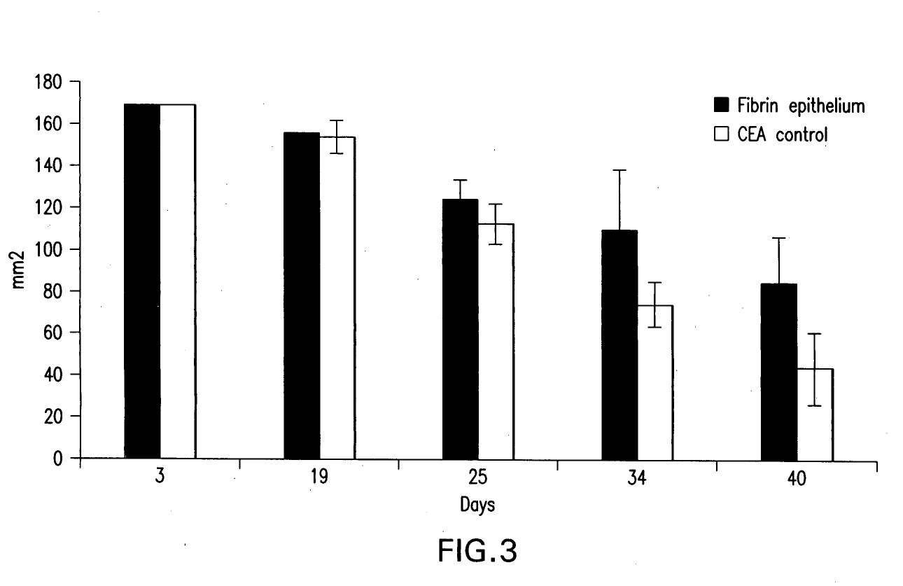

Human epidermis was identified at the wound site only in animals grafted with fibrin epithelium (5 out of 6 animals versus 0 out of 6 mice for CEA control (day 14 and 21 post-grafting) (FIG. 2)). The retention of the epithelial graft over time was also examined. FIG. 3 shows the size of the epithelial grafts in the presence or absence of a fibrin cell support on nude mice up to 40 days after grafting. As previously described, INTEGRA® was applied on all mice on day 0 and left undisturbed for 19 days. Then sheets of epithelium cultivated without fibrin (CEA control) or with fibrin (fibrin-epithelium) were applied onto INTEGRA®. The size of the graft was measured with a caliper and animals were sacrificed on day 40. Less retraction occurred when fibrin epithelium was applied onto INTEGRA®. A lower contraction rate of the initial wound size compared to animals grafted with CEA control or with no epithelial grafting was observed (FIG. 3). Therefore, the fibrin epithelium improved graft take, as compared to the control epithelial grafting method. This is supported by the increased cell survival shown above in in vitro studies.

### Example 6

### Method to Package the Fibrin Cell Support Containing Epithelial Cells

In order to facilitate the shipping and the transportation of the fibrin cell support containing epithelial cells, a fibrin cell support containing a confluent sheet of epithelium was rolled onto an inner plastic tube, and the fibrin cell support and plastic tube were inserted into an outer plastic tube. This outer tube was filed with medium, hermetically sealed and kept at room temperature for 48 hours. After this time, the stored epithelium were assayed for metabolic activity and viability (MTr test). The results demonstrated that metabolic activity is similar to the control fibrin epithelium not stored in this manner.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-42. (canceled)

43. A fibrin cell support for skin grafts, wherein said fibrin cell support is made by the process comprising:
forming a combined solution comprising thrombin and fibrinogen by mixing a first solution comprising thrombin with a second solution comprising fibrinogen, wherein the concentration of thrombin in said combined solution is from about 0.25 to about 2.5 U/ml;
distributing said combined solution onto a substrate before clotting of said combined solution occurs; and
allowing said distributed combined solution to clot and form a fibrin cell support.

44. The fibrin cell support of claim 43, wherein said concentration of thrombin in said combined solution is from about 0.5 to about 1.5 U/ml.

45. The fibrin cell support of claim 43, wherein the initial concentration of fibrinogen in said combined solution is from about 5 to about 125 mg/ml.

46. The fibrin cell support of claim 45, wherein the initial concentration of fibrinogen in said combined solution is from about 25 to about 75 mg/ml.

47. The fibrin cell support of claim 43, wherein said combined solution is distributed onto a support in the amount of about 50 μl of said solution comprising thrombin and fibrinogen per cm² of substrate surface area.

48. The fibrin cell support of claim 43 wherein said combined solution further comprises a protease inhibitor.

49. The fibrin cell support of claim 48, wherein said protease inhibitor is Aprotinin.

50. The fibrin cell support of claim 43, wherein said combined solution further comprises one or more molecules selected from the group consisting of a polypeptide growth factor, a cytokine, an enzyme, a hormone, an antibiotic, and an antimycotic, or a combination thereof.

51. The fibrin cell support of claim 43, wherein said fibrin cell support is made by the process further comprising seeding isolated cells onto said fibrin cell support.

52. The fibrin cell support of claim 51, wherein said isolated cells are keratinocytes.

53. The fibrin cell support of claim 51, wherein isolated cells are seeded at a subconfluent density.

54. The fibrin cell support of claim 53, wherein said isolated cells seeded at a subconfluent density are allowed to reach confluence in culture on said fibrin cell support.

55. The fibrin cell support of claim 43, wherein said wherein said combined solution comprising thrombin and fibrinogen further comprises isolated cells.

56. The fibrin cell support of claim 55, wherein said isolated cells are keratinocytes.

57. A method of using a fibrin skin graft comprising applying said fibrin skin graft to a patient in need of such treatment, wherein said fibrin skin graft was made by the process of:
forming a combined solution comprising thrombin and fibrinogen by mixing a first solution comprising thrombin with a second solution comprising fibrinogen, wherein the concentration of thrombin in said combined solution is from about 0.25 to about 2.5 U/ml;
distributing said combined solution onto a substrate before clotting of said combined solution occurs;
allowing said distributed combined solution to clot and form a fibrin cell support; and
seeding isolated cells onto said fibrin cell support.

58. The method of claim 57, wherein said concentration of thrombin in said combined solution is from about 0.5 to about 1.5 U/ml.

59. The method of claim 57, wherein the initial concentration of fibrinogen in said combined solution is from about 5 to about 125 mg/ml.

60. The method of claim 59, wherein the initial concentration of fibrinogen in said combined solution is from about 25 to about 75 mg/ml.

61. The method of claim 59, wherein said combined solution is distributed onto a support in the amount of about 50 μl of said solution comprising thrombin and fibrinogen per cm² of substrate surface area.

62. The method of claim 59, wherein said combined solution further comprises a protease inhibitor.

63. The method of claim 62 wherein said protease inhibitor is Aprotinin.

64. The method of claim 57, wherein said combined solution further comprises one or more molecules selected from the group consisting of a polypeptide growth factor, a cytokine, an enzyme, a hormone, an antibiotic, and an antimycotic, or a combination thereof.

65. The method of claim 57, wherein said isolated cells are autologous isolated cells.

66. The method of claim 57, wherein said isolated cells are keratinocytes.
